(19) United States
(12) Patent Application Publication
(10) Pub. No.: US 2014/0018687 A1
(43) Pub. Date: Jan. 16, 2014

(54) BLOOD PRESSURE MEASURING APPARATUS AND METHOD FOR CALIBRATING CENTRAL BLOOD PRESSURE ESTIMATION PARAMETER

(71) Applicant: Seiko Epson Corporation, Tokyo (JP)

(72) Inventor: Tomonori MANO, Matsumoto (JP)

(21) Appl. No.: 13/935,884

(22) Filed: Jul. 5, 2013

(30) Foreign Application Priority Data
Jul. 13, 2012 (JP) 2012-157225

(51) Int. Cl. A61B 5/021

(52) U.S. Cl. CPC A61B 5/021
USPC 600/485

(57) ABSTRACT

In an ultrasonic blood pressure meter 1, changes in blood pressure in a peripheral artery measured by a blood pressure meter 2 are input from an input unit 40. A blood vessel diameter measuring unit 120 measures changes in the blood vessel diameter of a central artery using ultrasound. A calibrating unit 130 calibrates a parameter related to a blood pressure estimation process for estimating central blood pressure from the blood vessel diameter of the central artery, using results of measurement by the blood pressure meter 2 and the blood vessel diameter measuring unit 120 during a given correspondence period, of a one-heartbeat period, in which the relationship between the blood vessel diameter of the central artery and the blood pressure in the peripheral artery corresponds to the relationship between the blood vessel diameter of the central artery and the central blood pressure.

### BACKGROUND

1. Technical Field

The present invention relates to blood pressure measuring apparatuses and the like that measure central blood pressure.

2. Related Art

In recent years, there have been devised an apparatus that measures blood flow, blood vessel diameter, and blood pressure using ultrasound, and an apparatus that measures the elastic modulus of a blood vessel using ultrasound. These apparatuses are characterized by their ability to perform noninvasive measurement that is painless and comfortable for subjects.

For example, JP-A-2004-41382 describes a method for estimating blood pressure from a stiffness parameter that indicates the stiffness of a blood vessel as well as the diameter or the cross-sectional area of the blood vessel, assuming that there is a nonlinear relationship between changes in the diameter of the blood vessel or changes in the cross-sectional area of the blood vessel and changes in the blood pressure.

JP-A-2004-41382 is an example of related art.

Now, it is considered that central blood pressure can be an index value of arteriosclerosis and circulatory diseases. When central blood pressure is estimated by applying the technology described in JP-A-2004-41382, it is necessary to measure blood pressure in a central artery such as the aorta or the carotid artery to calibrate the above-described stiffness parameter. However, measurement of blood pressure in the central arteries usually requires the use of an invasive method of measurement such as the insertion of a catheter, and thus there is a problem in that considerable physical stress is imposed on a subject.

Moreover, a blood pressure measuring apparatus that estimates central blood pressure from the waveform of blood pressure in, for example, the radial artery at the wrist has been put to practical use as an apparatus for measuring central blood pressure. However, since the radial artery is a peripheral artery, there are cases where central blood pressure cannot be accurately estimated.

### SUMMARY

An advantage of some aspects of the invention is to provide a novel method for central blood pressure measurement.

According to a first aspect of the invention for solving the above-described problems, a blood pressure measuring apparatus includes an input unit from which changes in blood pressure in a peripheral artery that are measured by a blood pressure measuring apparatus are input, a blood vessel cross section index value measuring unit that measures changes in a blood vessel cross section index value that is a blood vessel diameter or a blood vessel cross-sectional area of a central artery, and a calibrating unit that calibrates a parameter related to a blood pressure estimation process for estimating central blood pressure from the blood vessel cross section index value, using results of measurement by the blood pressure measuring apparatus and the blood vessel cross section index value measuring unit during a given correspondence period, of a one-heartbeat period, in which a relationship between the blood vessel cross section index value and the blood pressure in the peripheral artery corresponds to a relationship between the blood vessel cross section index value and the central blood pressure.

According to another aspect of the invention, a method for calibrating a central blood pressure estimation parameter may also be configured. The method includes measuring changes in blood pressure in a peripheral artery, measuring changes in a blood vessel cross section index value of a central artery, and calibrating a parameter related to a blood pressure estimation process for estimating central blood pressure from the blood vessel cross section index value, using measurement results of the blood pressure and the blood vessel cross section index value during a given correspondence period, of a one-heartbeat period, in which a relationship between the blood vessel cross section index value and the blood pressure in the peripheral artery corresponds to a relationship between the blood vessel cross section index value and the central blood pressure.

Although it is difficult to noninvasively measure blood pressure in a central artery, it is easy to noninvasively measure the blood vessel cross section index value of a central artery. For this reason, the central blood pressure can be estimated by performing the blood pressure estimation process for estimating the central blood pressure from the blood vessel cross section index value of the central artery. One of the features of this aspect is to calibrate a parameter related to the blood pressure estimation process. Usually, this calibration requires the blood pressure in a central artery. However, considering the difficulty of measurement of the blood pressure in the central artery, the blood pressure in a peripheral artery is used. Experiments made it clear that a one-heartbeat period contains a period in which the relationship between the blood vessel cross section index value of the central artery and the blood pressure in the peripheral artery corresponds to the relationship between the blood vessel cross section index value of the central artery and the central blood pressure. Therefore, the parameter related to the blood pressure estimation process is calibrated using measurement results of the blood pressure in the peripheral artery and the blood vessel cross section index value during this period. Thus, a parameter that is necessary for estimation of central blood pressure can be calibrated without the need for measuring blood pressure in a central artery. The central blood pressure can be correctly estimated by performing the blood pressure estimation process using the thus calibrated parameter.

Moreover, as a second aspect of the invention, the blood pressure measuring apparatus according to the first aspect may be configured so as to further include a first period setting unit that detects a diastolic period after a dicrotic wave peak from among the changes in the blood pressure that are input from the input unit, and sets the correspondence period so as to contain at least a part or the entirety of the diastolic period.

According to the second aspect, the diastolic period after the dicrotic wave peak is detected from among the changes in the blood pressure that are input from the input unit, and the correspondence period is set so as to contain at least a part or the entirety of the diastolic period. It became clear that during the diastolic period after the dicrotic wave peak, the relationship between the blood vessel cross section index value of the central artery and the blood pressure in the peripheral artery corresponds to the relationship between the blood vessel cross section index value of the central artery and the central blood pressure. Therefore, the parameter for estimating the central blood pressure can be appropriately calibrated by setting the correspondence period so as to contain at least a part or the entirety of that diastolic period.

Moreover, as a third aspect of the invention, the blood pressure measuring apparatus according to the first aspect may be configured so as to further include a second period setting unit that detects an ejection wave portion from among the changes in the blood pressure that are input from the input unit, and sets the correspondence period so as to contain at least a given rising period of the ejection wave portion.

According to the third aspect, the ejection wave portion is detected from among the changes in the blood pressure that are input from the input unit, and the correspondence period is set so as to contain at least a given rising period of that ejection wave portion. It became clear that during the blood pressure rising period of the ejection wave portion, the relationship between the blood vessel cross section index value of the central artery and the blood pressure in the peripheral artery corresponds to the relationship between the blood vessel cross section index value of the central artery and the central blood pressure. Therefore, the parameter for estimating the central blood pressure can be appropriately calibrated by setting the correspondence period so as to contain at least this rising period.

Moreover, as a fourth aspect of the invention, the blood pressure measuring apparatus according to the third aspect may be configured such that the second period setting unit performs setting such that the rising period contains at least a period from a start of rising of the ejection wave portion to when ⅕ of the ejection wave portion elapses.

According to the fourth aspect, setting is performed such that the rising period contains at least a period from the start of rising of the ejection wave portion to when ⅕ of the ejection wave portion elapses. The reason for this is that during at least a period of about ⅕ of the ejection wave portion, the relationship between the blood vessel cross section index value of the central artery and the blood pressure in the peripheral artery can be regarded as the relationship between the blood vessel cross section index value of the central artery and the central blood pressure.

Moreover, as a fifth aspect of the invention, the blood pressure measuring apparatus according to the first aspect may be configured so as to further include a synchronizing unit that synchronizes the changes in the blood pressure that are input from the input unit with the changes in the blood vessel cross section index value that are measured by the blood vessel cross section index value measuring unit, wherein the calibrating unit calibrates the parameter using measurement results of the blood pressure and the blood vessel cross section index value that are synchronized by the synchronizing unit.

Since a central artery and a peripheral artery are different from each other in terms of the distance and the route from the heart, the times when a blood flow arrives at these arteries after being ejected from the heart are different (delay in pulse wave propagation). To address this issue, as in the fifth aspect, the changes in the blood pressure that are input from the input unit are synchronized with the changes in the blood vessel cross section index value that are measured by the blood vessel cross section index value measuring unit. The parameter can be accurately calibrated by using the measurement results of the blood pressure and the blood vessel cross section index value that are synchronized.

### BRIEF DESCRIPTION OF THE DRAWINGS

The invention will be described with reference to the accompanying drawings, wherein like numbers reference like elements.

FIG. 1 is an explanatory diagram of correlation characteristics that carotid artery diameter has with carotid artery blood pressure and radial artery blood pressure.

FIG. 2 is a diagram illustrating an example of changes in the radial artery blood pressure and the carotid artery diameter with time.

FIG. 3 is a graph illustrating an example of changes in the radial artery blood pressure versus changes in the carotid artery diameter.

FIG. 4 is a diagram schematically illustrating the configuration of an ultrasonic blood pressure meter.

FIG. 5 is a block diagram illustrating an example of the functional configuration of the ultrasonic blood pressure meter.

FIG. 6 is a flowchart illustrating the flow of a calibration process.

FIG. 7 is a flowchart illustrating the flow of a second calibration process.

### DESCRIPTION OF EXEMPLARY EMBODIMENTS

The following describes an example of a preferred embodiment to which the invention is applied, with reference to the drawings. In this embodiment, blood vessel diameter will be described as a blood vessel cross section index value. However, it is also possible to use blood vessel cross-sectional area instead of blood vessel diameter (in this case, “blood vessel diameter” in the following description can be replaced with “blood vessel cross-sectional area”). It goes without saying that embodiments to which the invention is applicable are not limited to the embodiment that will be described below.

### 1. Principle

In this embodiment, a blood pressure measuring apparatus (central blood pressure measuring apparatus) that measures central blood pressure is calibrated by specifying a parameter related to a blood pressure estimation process for estimating central blood pressure (hereinafter referred to as “central blood pressure estimation parameter”). In this specification, specifying the value of the central blood pressure estimation parameter will be referred to as calibration of the central blood pressure estimation parameter.

Central blood pressure refers mainly to the blood pressure in a beginning portion of the aorta, which is one of the central arteries. There also are cases where blood pressure in the carotid artery (hereinafter referred to as “carotid artery blood pressure”) is regarded as central blood pressure. In this embodiment, the blood vessel diameter of a central artery (hereinafter referred to as “central artery diameter”) is measured, and central blood pressure is estimated by performing a predetermined blood pressure estimation process using the measured central artery diameter and the value of the calibrated central blood pressure estimation parameter.

In order to estimate blood pressure from blood vessel diameter, it is necessary to use correlation characteristics that link the blood vessel diameter with the blood pressure. It is possible to link the blood vessel diameter with the blood pressure using, for example, certain nonlinear correlation characteristics. Specifically, the correlation characteristics can be expressed by a correlation formula, such as Formula 1 below, by using a pressure applied to a blood vessel and a blood vessel diameter at each blood pressure period:

P=Pd·exp[β(D/Dd−1)] (1)

where β=ln(Ps/Pd)/(Ds/Dd−1).

Here, “Ps” represents systolic blood pressure (maximum blood pressure), and “Pd” represents diastolic blood pressure (minimum blood pressure). Moreover, “Ds” represents systolic blood vessel diameter, which is the blood vessel diameter when the blood pressure is the systolic blood pressure, and “Dd” represents diastolic blood vessel diameter, which is the blood vessel diameter when the blood pressure is the diastolic blood pressure. Moreover, “β” represents a blood vessel elasticity index value that is called a stiffness parameter.

In the blood pressure estimation process, central blood pressure is estimated by applying the correlation formula of Formula 1 to a central artery. That is to say, the blood pressure “P” is estimated by substituting the central artery diameter for the blood vessel diameter “D” in Formula 1. The blood pressure “P” that is thus estimated is regarded as the blood pressure in the central artery, that is, the central blood pressure. Any appropriate method can be chosen as the method for measuring the central artery diameter. For example, a method that measures the blood vessel diameter using ultrasound can be applied.

In order to estimate central blood pressure using Formula 1, the value of the above-described stiffness parameter “β” is necessary. In the description of this embodiment, this stiffness parameter “β” is used as the parameter related to the blood pressure estimation process for estimating central blood pressure (hereinafter referred to as “central blood pressure estimation parameter”).

In the following description, the diameter of the carotid artery (hereinafter referred to as “carotid artery diameter”) is used as the central artery diameter, and blood pressure in the carotid artery (hereinafter referred to as “carotid artery blood pressure”) is used as the central artery blood pressure. Moreover, blood pressure in the radial artery at the wrist (hereinafter referred to as “radial artery blood pressure”) is used as the peripheral artery blood pressure.

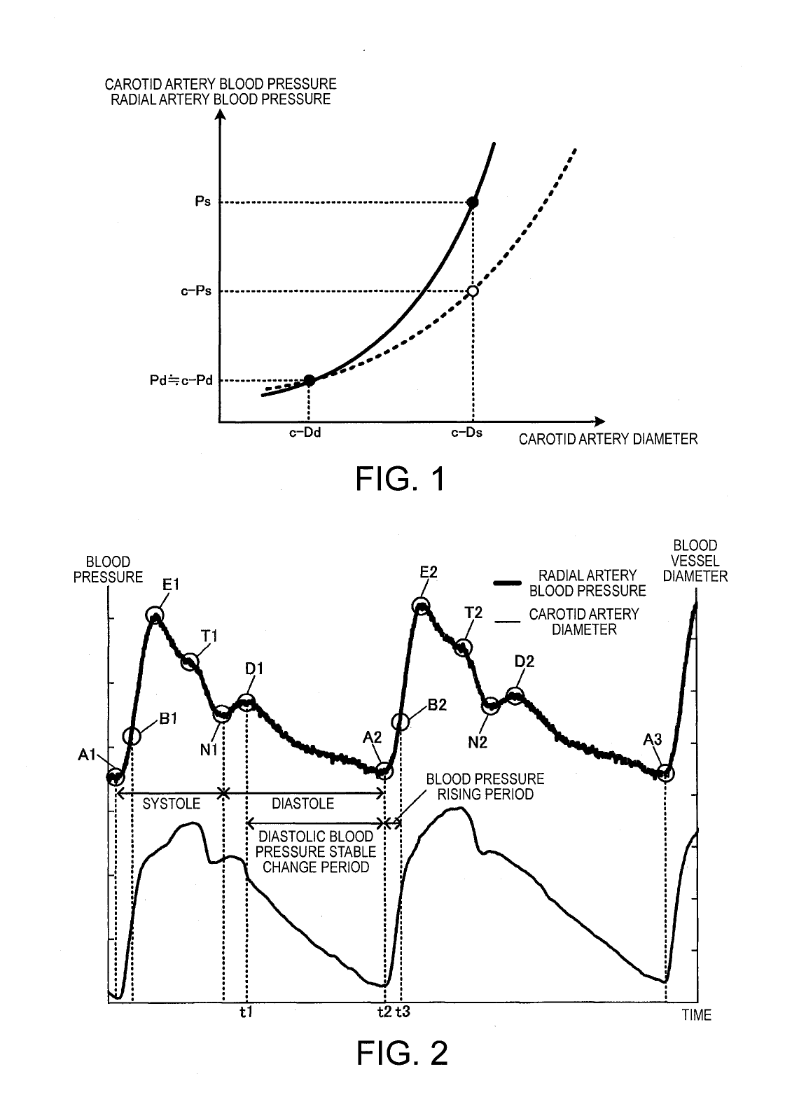

FIG. 1 is an explanatory diagram of correlation characteristics between blood vessel diameter and blood pressure. In FIG. 1, the horizontal axis indicates the carotid artery diameter, and the vertical axis indicates the carotid artery blood pressure and the radial artery blood pressure. With regard to the carotid artery diameter, the carotid artery diameter during diastole (hereinafter referred to as “diastolic carotid artery diameter”) is denoted by “c-Dd”, and the carotid artery diameter during systole (hereinafter referred to as “systolic carotid artery diameter”) is denoted by “c-Ds”. Moreover, the carotid artery blood pressure during diastole (hereinafter referred to as “diastolic carotid artery blood pressure”) is denoted by “c-Pd”, and the carotid artery blood pressure during systole (hereinafter referred to as “systolic carotid artery blood pressure”) is denoted by “c-Ps”. Furthermore, the radial artery blood pressure during diastole (hereinafter referred to as “diastolic radial artery blood pressure”) is denoted by “Pd”, and the radial artery blood pressure during systole (hereinafter referred to as “systolic radial artery blood pressure”) is denoted by “Ps”.

In the diagram, coordinate values that are defined by the carotid artery diameter and the carotid artery blood pressure are plotted as hollow circles, and coordinate values that are defined by the carotid artery diameter and the radial artery blood pressure are plotted as solid circles. This diagram shows that the radial artery diastolic blood pressure Pd and the carotid artery diastolic blood pressure c-Pd are approximately equal values, but the radial artery systolic blood pressure Ps and the carotid artery systolic blood pressure c-Ps are considerably different values. Although there are differences among individuals, the carotid artery systolic blood pressure c-Ps tends to be lower than the radial artery systolic blood pressure Ps by up to about 20 mmHg. This can be attributed to a so-called peaking phenomenon or the influence of a reflected wave.

Since it is not easy to noninvasively measure the carotid artery blood pressure, consideration is given to substitution of the radial artery blood pressure for calibration of the central blood pressure estimation parameter. In this case, a correlation formula as shown by the solid line can be acquired by obtaining the value of the stiffness parameter “β” in Formula 1 using the two points indicated by the solid circles. However, actually, the correlation between the carotid artery diameter and the carotid artery blood pressure is expressed by a correlation formula as shown by the dotted line and is not consistent with the correlation formula shown by the solid line. That is to say, the obtained correlation between the carotid artery diameter and the carotid artery blood pressure would not be correct, and estimating the central blood pressure based on this correlation would cause an error in the estimated central blood pressure.

Thus, in this embodiment, attention is paid to the presence of a period in which the relationship between the central artery diameter and the peripheral artery blood pressure corresponds to the relationship between the central artery diameter and the central blood pressure, and the central blood pressure estimation parameter is calibrated using the measurement results of the peripheral artery blood pressure and the central artery diameter during that period.

FIG. 2 is a diagram illustrating an example of the results of an experiment in which waveforms of changes in blood pressure and blood vessel diameter are measured. The horizontal axis is a time axis, and the vertical axis indicates the radial artery blood pressure and the carotid artery diameter. This graph illustrates changes in the radial artery blood pressure and changes in the carotid artery diameter over two heartbeats.

The waveform of the radial artery blood pressure shows that there are three main types of peaks of the blood pressure. The peaks of the first type are the peaks (hereinafter referred to as “ejection wave peaks”) E (E1, E2) of an ejection wave that are observed as a result of an ejection wave being emitted when the aortic valve opens, and portions of the radial artery blood pressure waveform in the diagram at which the blood pressure is maximized correspond to this type of peak.

The peaks of the second type are the peaks (hereinafter referred to as “tidal wave peaks”) T (T1, T2) of a tidal wave (ebbing wave), which is a reflected wave from an arterial bifurcation, and small peaks that are observed first after the ejection wave peaks E in the radial artery blood pressure waveform in the diagram correspond to this type of peak.

The peaks of the third type are the peaks (hereinafter referred to as “dicrotic wave peaks”) D (D1, D2) of a dicrotic wave (double wave), which is a reflected wave after closure of the aortic valve. In the radial artery blood pressure waveform in this diagram, the peaks that are observed immediately after notches N (N1, N2) correspond to this type of peak.

By common definition, “systole” is the period from when the aortic valve opens to when the aortic valve closes, and “diastole” is the period from when the aortic valve closes to when the aortic valve opens the next time. Thus, in FIG. 2, systole and diastole are shown so as to correspond to the radial artery blood pressure waveform. A period consisting of a systole and a diastole is defined as “one-heartbeat period”. Moreover, a portion of the blood pressure waveform from a diastolic blood pressure to an ejection wave peak is defined as “ejection wave portion”.

The description below focuses on the waveform of the radial artery blood pressure in FIG. 2. In a one-heartbeat period of the first heartbeat, blood is ejected from the heart in conjunction with opening of the aortic valve, and the blood pressure sharply rises from the diastolic blood pressure A1. Then, the ejection wave peak E1 is observed at the highest point of the blood pressure. After that, the blood pressure begins decreasing, but the tidal wave peak T1 is observed due to the effect of the tidal wave, which is a reflected wave from an arterial bifurcation.

Then, the blood pressure decreases again, and the notch N1 is observed when the aortic valve closes. A notch corresponds to the end of systole. Then, a dicrotic wave, which is a reflected oscillatory wave, occurs as a result of the aortic pressure causing blood flow to rush to the aortic valve. This results in a temporary increase in the blood pressure, and thus the dicrotic wave peak D1 is observed. After that, the blood pressure gently decreases and reaches the diastolic blood pressure A2 of the next heartbeat. The same applies to the second and subsequent heartbeats.

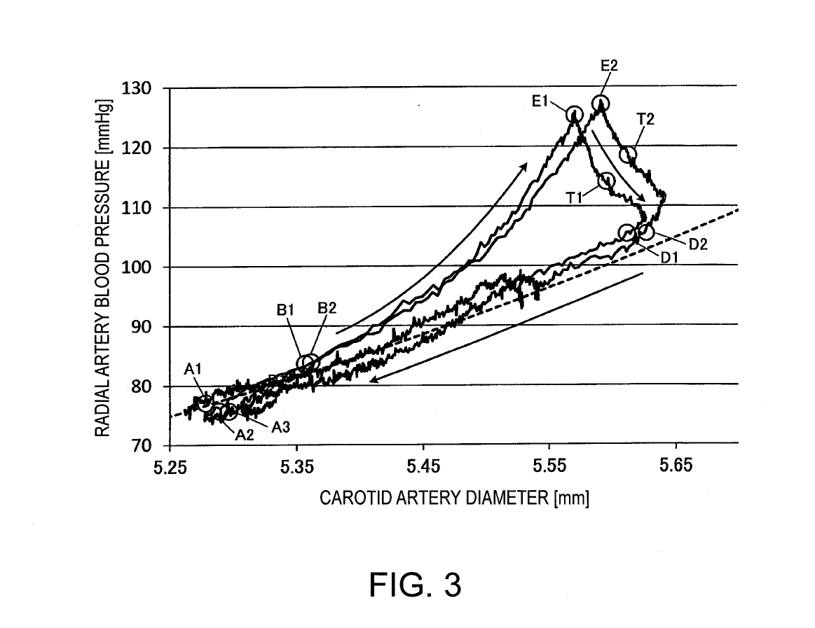

FIG. 3 is an example of a graph that shows the relationship between the carotid artery diameter and the radial artery blood pressure in correspondence with the waveform in FIG. 2. The graph, in which the carotid artery diameter is indicated by the horizontal axis and the radial artery blood pressure is indicated by the vertical axis, shows an example of the results of observation on how the radial artery blood pressure changes with changes in the carotid artery diameter. The graph shown here corresponds to the waveform over two heartbeats in FIG. 2. Moreover, a correlation formula (in a sense, an ideal correlation formula) that expresses the correlation between the carotid artery diameter and the carotid artery blood pressure is shown by the dotted line.

It can be seen from this diagram that considerable hysteresis is exhibited because the blood pressure measurement site (at the wrist) is different from the blood vessel diameter measurement site (at the neck). The changes in the radial artery blood pressure with respect to the carotid artery diameter forms a substantially right triangular shape. When attention is focused on the correspondence relationship in the portion corresponding to the first heartbeat, the diastolic blood pressure A1 is located in the lower-left portion. The radial artery blood pressure sharply rises with the increase in the carotid artery diameter, and the blood pressure reaches a maximum at the ejection wave peak E1.

After reaching the ejection wave peak E1, the radial artery blood pressure gradually decreases, whereas the carotid artery diameter increases because this phase is included in systole. The blood pressure shifts from the ejection wave peak E1 via the tidal wave peak T1 to the dicrotic wave peak D1. In this process, transition from systole to diastole occurs due to closure of the aortic valve, and therefore the carotid artery diameter decreases with the radial artery blood pressure. Then, finally, the radial artery blood pressure reaches the diastolic blood pressure A2 of the second heartbeat.

It can be seen that, in FIG. 3, the considerable hysteresis is exhibited almost entirely during systole. In contrast, during the period from a dicrotic wave peak to the diastolic blood pressure of the next heartbeat, the carotid artery diameter and the radial artery blood pressure change in conformity with the ideal correlation formula shown by the dotted line. That is to say, it can be said that the period from a dicrotic wave peak to the diastolic blood pressure of the next heartbeat is a period in which changes in the radial artery blood pressure with respect to changes in the carotid artery diameter can be regarded as changes in the central blood pressure with respect to the changes in the carotid artery diameter. This period is a period of diastole in which the blood pressure changes stably, and so this period will be referred to as “diastolic blood pressure stable change period” in the following description.

In addition to this, it can be seen that during a period in which the blood pressure rises from the diastolic blood pressure to an ejection wave peak, for a predetermined period after the start of rising of the blood pressure, the carotid artery diameter and the radial artery blood pressure change in conformity with the ideal correlation formula shown by the dotted line. This period is a rising period of the blood pressure of the ejection wave portion, and so this period will be referred to as “blood pressure rising period” in the following description.

Moreover, the blood pressure rising period can be defined as, for example, a period from the start of rising of an ejection wave portion to when ⅕ to ⅓ of that ejection wave portion elapses. When attention is focused on changes in the radial artery blood pressure of the first heartbeat in the graph of FIG. 3, the carotid artery diameter corresponding to the diastolic blood pressure A1 is a little more than 5.25 mm, and the carotid artery diameter corresponding to the ejection wave peak E1 is a little more than 5.55 mm. Accordingly, the difference between the carotid artery diameter at the diastolic blood pressure A1 and the carotid artery diameter at the ejection wave peak E1 is about 0.3 mm.

Meanwhile, changes conforming to the ideal correlation formula shown by the dotted line can be observed between the diastolic blood pressure A1 and the portion of the blood pressure that corresponds to a carotid artery diameter of about 5.35 mm. Accordingly, during a period from the start of rising of an ejection wave portion until at least ⅓ of that ejection wave portion elapses, changes in the radial artery blood pressure with respect to changes in the carotid artery diameter can be regarded as changes in the central blood pressure with respect to the changes in the carotid artery diameter. It should be noted that any period in which the same correspondence relationship is exhibited can be used as this period, and so this period may also be, for example, until “⅕” of the ejection wave portion elapses, instead of “⅓”. However, “⅓” is employed in the description of this embodiment.

Referring again to FIG. 2, a period between times t1 and t2 corresponds to the diastolic blood pressure stable change period, and a period between times t2 and t3 corresponds to the blood pressure rising period. Moreover, the blood pressures indicated by B1 for the first heartbeat and B2 for the second heartbeat are the blood pressures corresponding to the ends of respective blood pressure rising periods.

In this embodiment, any period among (A) the diastolic blood pressure stable change period, (B) the blood pressure rising period, and (C) the diastolic blood pressure stable change period+the blood pressure rising period is set as a calibration data acquisition period. Then, the central blood pressure estimation parameter is calibrated using the measurement results of the peripheral artery blood pressure and the central artery diameter during the set calibration data acquisition period.

In this case, several methods are conceivable as the method for calibrating the central blood pressure estimation parameter. One of these methods is a method in which the value of the stiffness parameter “β” is specified by fitting a function that is defined by Formula 1 to measurement data during the calibration data acquisition period using, for example, the least-squares method.

Another conceivable method is a method in which the measurement data during the calibration data acquisition period is classified on the basis of blood pressure magnitude, and the value of the stiffness parameter “β” is specified using the classified measurement data. More specifically, a predetermined threshold blood pressure (for example, 90 mmHg) is set, and the measurement data during the calibration data acquisition period is divided into two measurement data groups according to magnitude relative to the threshold blood pressure. Then, for each of the two measurement data groups, an averaging process is performed on the measurement data for each of the two measurement data groups to calculate mean values of the blood pressure and the blood vessel diameter. Then, with respect to the calculated mean values of the blood pressure and the blood vessel diameter related to the two data groups, Formula 1 is simultaneously solved, and thus the value of the stiffness parameter “β” is calculated and specified.

It should be noted that although the method of calibration according to this embodiment can theoretically calibrate the central blood pressure estimation parameter with measurement data over a single heartbeat, it is also possible to obtain the value of the central blood pressure estimation parameter using measurement data over a plurality of heartbeats.

Specifically, for example, with respect to a predetermined number of consecutive heartbeats (e.g., thirty heartbeats), a calibration data acquisition period is set for each one-heartbeat period. Then, measurement data for use in calibration of the central blood pressure estimation parameter is specified by, for example, statistically processing measurement data during the set calibration data acquisition period, and the central blood pressure estimation parameter can be calibrated using the specified measurement data.

### 2. Example

Next, an example of a blood pressure measuring apparatus that measures central blood pressure according to the above-described principle will be described using the radial artery of the subject as the peripheral artery and the carotid artery as the central artery. The blood pressure measuring apparatus of this example is an ultrasonic blood pressure meter that measures central blood pressure using ultrasound.

2-1. Schematic Configuration

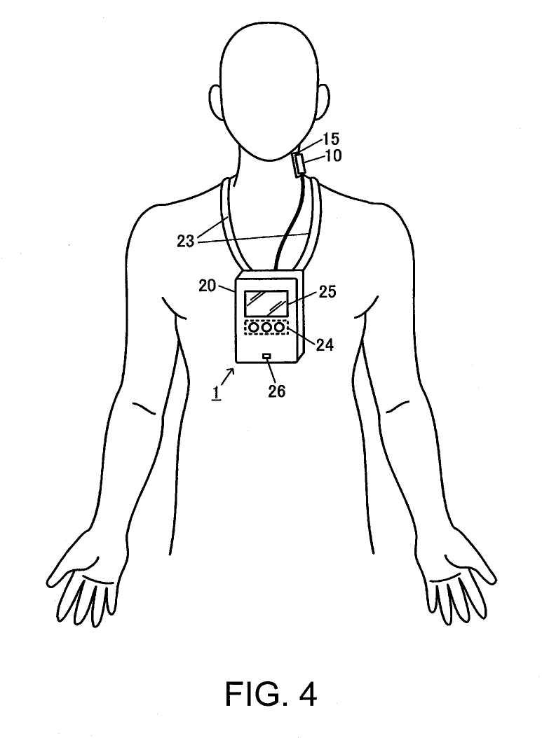

FIG. 4 is a diagram schematically illustrating the configuration of an ultrasonic blood pressure meter 1 of this example. The ultrasonic blood pressure meter 1 has an ultrasound probe 10 and a body apparatus 20. The subject wears the ultrasonic blood pressure meter 1 using an attachment tape 15 so that the ultrasound probe 10 is located over the carotid artery.

The ultrasound probe 10 transmits an ultrasound pulse signal or burst signal of several MHz to several tens MHz from a transmitting unit toward the carotid artery. Then, a receiving unit of the ultrasound probe 10 receives reflected waves from an anterior wall and a posterior wall of the carotid artery, and the carotid artery diameter is measured as a blood vessel cross section index value from the difference in receiving time between the reflected wave from the anterior wall and the reflected wave from the posterior wall.

The body apparatus 20 is an apparatus main body of the ultrasonic blood pressure meter 1, and is connected to the ultrasound probe 10 in a wired manner via a cable. A neck strap 23 for enabling the subject to hang the body apparatus 20 around the neck during usage is attached to the body apparatus 20.

Operating buttons 24, a liquid crystal display 25, and a speaker 26 are provided on a front surface of the body apparatus 20. Moreover, although not shown in the drawing, a control board for performing integrated control of the devices is built into the body apparatus 20. A microprocessor, a memory, a circuit related to ultrasound transmission and reception, an internal battery, and the like are mounted on the control board.

The operating buttons 24 are used by a user to input an instruction to start central blood pressure measurement and various amounts related to central blood pressure measurement.

The results of central blood pressure measurement by the ultrasonic blood pressure meter 1 are displayed on the liquid crystal display 25. With regard to the method of display, measured values of central blood pressure may be numerically displayed or may be displayed in the form of a graph or the like.

Moreover, various types of audio guidance and the like related to central blood pressure measurement are output from the speaker 26.

In this example, the ultrasonic blood pressure meter 1 is calibrated using the results of radial artery blood pressure measurement by a blood pressure meter 2 that is prepared separately from the ultrasonic blood pressure meter 1. The blood pressure meter 2 is a blood pressure measuring apparatus that is capable of continuous blood pressure measurement, and may be a tonometer that measures changes in blood pressure using tonometry, for example. Tonometry is a technique for measurement of changes in blood pressure by pressing a sensor having a flat contact surface against an artery to be measured and converting fluctuations of internal pressure of the artery that pulsates against this pressure into an electric signal.

2-2. Functional Configuration

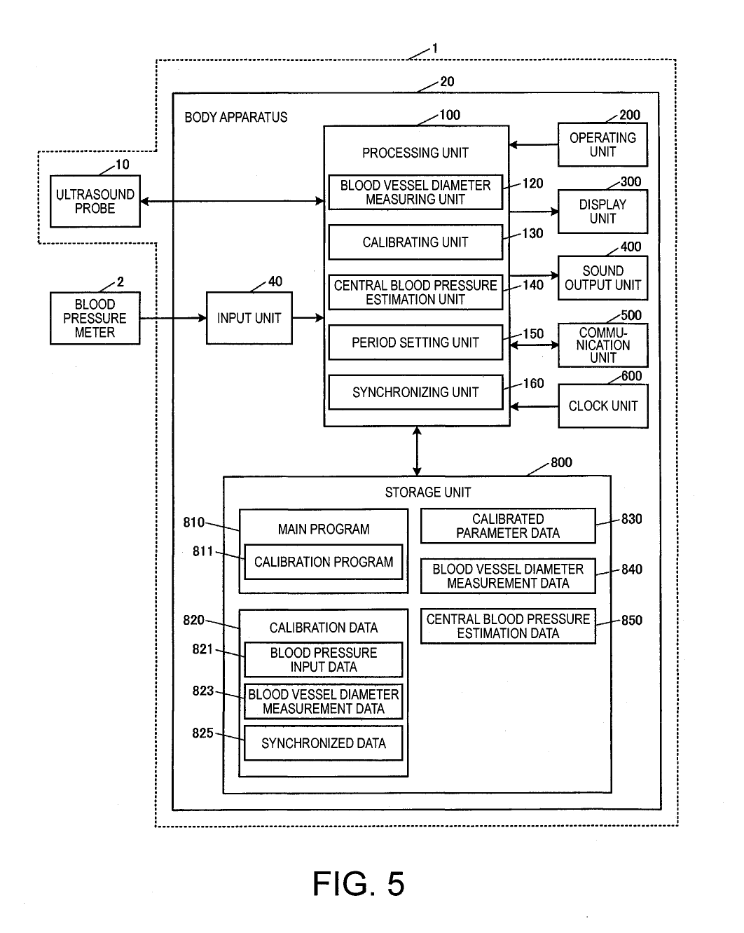

FIG. 5 is a block diagram showing an example of the functional configuration of the ultrasonic blood pressure meter 1. The ultrasonic blood pressure meter 1 has the ultrasound probe 10 and the body apparatus 20, and is configured so as to be connected to the blood pressure meter 2 by cable so that the results of radial artery blood pressure measurement can be input from the blood pressure meter 2.

The ultrasound probe 10 is a small-size contact that performs ultrasound transmission and reception by switching between an ultrasound transmission mode and an ultrasound reception mode in a time-division manner, in accordance with a control signal from a blood vessel diameter measuring unit 120. A received signal is output to the blood vessel diameter measuring unit 120.

The body apparatus 20 has an input unit 40, a processing unit 100, an operating unit 200, a display unit 300, a sound output unit 400, a communication unit 500, a clock unit 600, and a storage unit 800.

The input unit 40 is an interface which is connected to the blood pressure meter 2 and from which the results of blood pressure measurement from the blood pressure meter 2 are input. The input unit 40 corresponds to an input unit from which changes in blood pressure in a peripheral artery measured by a blood pressure measuring apparatus are input.

The processing unit 100 is a control unit and arithmetic unit that performs integrated control of various units of the ultrasonic blood pressure meter 1, and has microprocessors such as a CPU (Central Processing Unit) and a DSP (Digital Signal Processor), an ASIC (Application Specific Integrated Circuit), and the like.

The processing unit 100 has as its main functional units the blood vessel diameter measuring unit 120, a calibrating unit 130, a central blood pressure estimation unit 140, a period setting unit 150, and a synchronizing unit 160. However, these functional units are mentioned as examples only, and it is not necessarily required that all of these functional units are provided as essential components. Moreover, it goes without saying that any functional unit other than these may also be provided as an essential component.

The blood vessel diameter measuring unit 120 controls ultrasound transmission and reception of the ultrasound probe 10, and measures the blood vessel diameter of a target blood vessel using a received signal of a ultrasonic reflected wave that is output from the ultrasound probe 10. In this embodiment, the carotid artery (e.g., the common carotid artery), which is one of the central arteries, serves as the target blood vessel. The blood vessel diameter measuring unit 120 corresponds to a blood vessel cross section index value measuring unit that measures changes in a blood vessel cross section index value of a central artery.

The blood vessel diameter measuring unit 120 is a measuring unit that measures changes in the blood vessel diameter of a target artery. In this embodiment, the blood vessel diameter measuring unit 120 measures changes in carotid artery diameter by continuously measuring the blood vessel diameter using a phase difference tracking method. It should be noted that since the phase difference tracking method is previously known, a description of details of this method is not given here.

The calibrating unit 130 calibrates the central blood pressure estimation parameter using the results of radial artery blood pressure measurement that have been input from the input unit 40 and the results of carotid artery diameter measurement by the blood vessel diameter measuring unit 120.

The central blood pressure estimation unit 140 estimates the central blood pressure by executing a blood pressure estimation process for estimating the central blood pressure from the blood vessel diameter that has been measured by the blood vessel diameter measuring unit 120.

The period setting unit 150 sets a calibration data acquisition period in accordance with the above-described principle. The period setting unit 150 corresponds to a first period setting unit and a second period setting unit.

The synchronizing unit 160 synchronizes changes in blood pressure that have been input from the input unit 40 with changes in blood vessel diameter that have been measured by the blood vessel diameter measuring unit 120. Since the carotid artery and the radial artery are different from each other in terms of the distance and the route from the heart, the times when a blood flow arrives at these arteries after being ejected from the heart are different (delay in pulse wave propagation). Thus, the changes in blood pressure that have been input from the blood pressure meter 2 and the changes in blood vessel diameter that have been measured by the blood vessel diameter measuring unit 120 cannot be compared as they are, and it is necessary to perform time alignment therebetween. For this reason, the synchronizing unit 160 executes a synchronization process for synchronizing the changes in blood pressure with the changes in blood vessel diameter as preprocessing.

The operating unit 200 is an input device that has a button switch and the like, and outputs a signal corresponding to a button that has been pressed to the processing unit 100. Various instructions such as an instruction to start central blood pressure measurement are input by operating this operating unit 200. The operating unit 200 corresponds to the operating buttons 24 in FIG. 4.

The display unit 300, which has an LCD (Liquid Crystal Display) and the like, is a display device that performs various types of display based on a display signal input from the processing unit 100. Information on the central blood pressure estimated by the central blood pressure estimation unit 140 and so on is displayed on the display unit 300. The display unit 300 corresponds to the liquid crystal display 25 in FIG. 4.

The sound output unit 400 is a sound output device that performs various types of sound output based on a sound output signal input from the processing unit 100. For example, the sound output unit 400 outputs sounds that announce the start of measurement, the end of measurement, the occurrence of an error, and the like. The sound output unit 400 corresponds to the speaker 26 in FIG. 4.

The communication unit 500 is a communication device for transmitting and receiving information that is used inside the apparatus to and from an outside information processing apparatus as controlled by the processing unit 100. With regard to the communication system of the communication unit 500, various systems are applicable including a form in which wired connection is achieved via a cable that conforms to a predetermined communication standard, a form in which connection is achieved via an intermediate device that is called a cradle and that doubles as a charger, a form in which wireless connection is achieved using near field communication, and the like. In the case where the blood pressure meter 2 is connected by communication connection, the input unit 40 serves as the communication unit 500.

The clock unit 600 is a time measuring device that has a crystal oscillator constituted by a crystal unit and an oscillation circuit, and the like, and that measures time. The time measured by the clock unit 600 is output to the processing unit 100 at any time.

The storage unit 800 has storage devices such as a ROM (Read Only Memory), a flash ROM, and a RAM (Random Access Memory). The storage unit 800 stores a system program of the ultrasonic blood pressure meter 1, various programs for realizing various functions such as a calibration function and a central blood pressure estimation function, data, and the like. Moreover, the storage unit 800 has a work area that temporarily stores mid-process data, processing results, and the like of various processes.

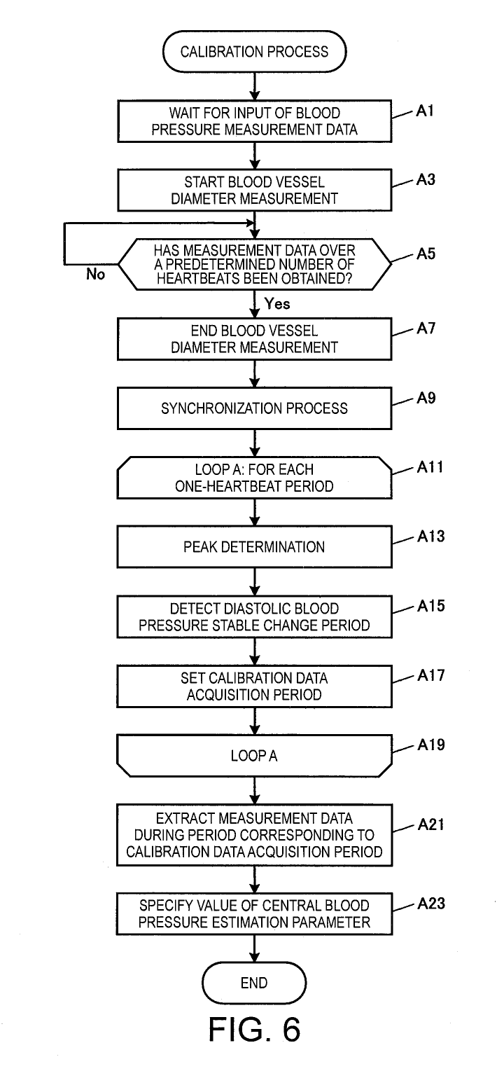

A main program 810 that is read out and executed as a main process by the processing unit 100 is an example of the programs stored in the storage unit 800. The main program 810 contains a calibration program 811 as a subroutine, the calibration program 811 being executed as a calibration process (see FIG. 6). The calibration process will be described in detail later using a flowchart.

Moreover, the storage unit 800 stores, as the data, calibration data 820, calibrated parameter data 830, blood vessel diameter measurement data 840, and central blood pressure measurement data 850.

The calibration data 820 is data that is used for calibration of the central blood pressure estimation parameter, and contains blood pressure input data 821, blood vessel diameter measurement data 823, and synchronized data 825.

As the blood pressure input data 821, measured values of blood pressure that have been input from the blood pressure meter 2 via the input unit 40 are stored in association with time.

As the blood vessel diameter measurement data 823, measured values of blood vessel diameter that have been measured by the blood vessel diameter measuring unit 120 are stored in association with time.

The synchronized data 825 is data on the blood pressure and the blood vessel diameter that have been synchronized by the synchronizing unit 160.

As the calibrated parameter data 830, the value of the central blood pressure estimation parameter that has been calibrated by the calibrating unit 130 is stored. For example, the calibrated parameter data 830 contains the value of the stiffness parameter “β” in Formula 1.

As the blood vessel diameter measurement data 840, measured values of blood vessel diameter that have been measured by the blood vessel diameter measuring unit 120 in normal measurement are stored.

As the central blood pressure measurement data 850, estimated values of the central blood pressure that have been estimated by the central blood pressure estimation unit 140 in normal measurement are stored.

2-3. Processing Flow

FIG. 6 is a flowchart illustrating the flow of the calibration process that is executed by the processing unit 100 in accordance with the calibration program 811 stored in the storage unit 800. The processing unit 100 performs the calibration process at the time of initial measurement or at regular timings in the main process that the processing unit 100 executes in accordance with the main program 810.

The processing unit 100 waits for input of blood pressure measurement data from the blood pressure meter 2 (step A1). When input of the blood pressure measurement data is started, the processing unit 100 stores the blood pressure measurement data that has been input from the input unit 40 in the calibration data 820 as the blood pressure input data 821.

In response to the input of the blood pressure measurement data, the blood vessel diameter measuring unit 120 starts blood vessel diameter measurement (step A3). Specifically, changes in the blood vessel diameter of the carotid artery are measured using the phase difference tracking method, and the measurement data on the blood vessel diameter is stored in the calibration data 820 as the blood vessel diameter measurement data 823.

The processing unit 100 waits until measurement data over a predetermined number of heartbeats is obtained (step A5: No). Then, if the measurement data over the predetermined number of heartbeats is obtained (step A5: Yes), the blood vessel diameter measuring unit 120 ends the blood vessel diameter measurement (step A7).

Subsequently, the synchronizing unit 160 performs the synchronization process (step A9). Specifically, time alignment between the blood pressures stored in the blood pressure input data 821 and the blood vessel diameters stored in the blood vessel diameter measurement data 823 is performed. Specifically, the two kinds of measurement data are synchronized with each other by, for example, aligning the times so that the diastolic blood vessel diameters (minimum blood vessel diameters) correspond to the diastolic blood pressures (minimum blood pressures).

Subsequently, the processing unit 100 performs processing of a loop A for each one-heartbeat period (steps A11 to A19). In the processing of the loop A, the processing unit 100 determines peaks of the changes in the blood pressure during the relevant one-heartbeat period (step A13). Then, a diastolic blood pressure stable change period is detected based on the determined peaks. That is to say, a diastolic period after a dicrotic wave peak within diastole is detected as the diastolic blood pressure stable change period.

Subsequently, the period setting unit 150 sets a calibration data acquisition period based on the diastolic blood pressure stable change period that was detected in step A15 (step A17). Specifically, the calibration data acquisition period is set so as to contain the entirety or a part of the diastolic blood pressure stable change period. For simplification, the diastolic blood pressure stable change period can be set as the calibration data acquisition period as it is. Then, the processing unit 100 advances the processing to the subsequent one-heartbeat period. When the processing of steps A13 to A17 has been performed for all of the one-heartbeat periods, the processing unit 100 ends the processing of the loop A (step A19).

After that, the processing unit 100 extracts, from the synchronized data 825, the measurement data on the blood pressure and the blood vessel diameter during a period corresponding to the calibration data acquisition period that was set in step A17 for each one-heartbeat period (step A21). Then, the calibrating unit 130 calculates and specifies the value of the central blood pressure estimation parameter using the extracted measurement data, and stores this value in the storage unit 800 as the calibrated parameter data 830 (step A23). Then, the processing unit 100 ends the calibration process.

After the calibration process in FIG. 6 is performed, the blood pressure meter 2 is detached, and the measurement proceeds to normal measurement. In normal measurement, the blood vessel diameter measuring unit 120 measures the carotid artery diameter, and stores the measurement results in the blood vessel diameter measurement data 840. The central blood pressure estimation unit 140 estimates the carotid artery blood pressure as the central blood pressure using a correlation formula that is defined by the value of the central blood pressure estimation parameter stored in the calibrated parameter data 830 as well as the carotid artery diameter measured by the blood vessel diameter measuring unit 120, and stores the estimate in the central blood pressure estimation data 850. Then, the subject is informed of the estimated central blood pressure by means of, for example, display on the display unit 300.

2-4. Effects

In the ultrasonic blood pressure meter 1, changes in the blood pressure in the peripheral artery that have been measured by the blood pressure meter 2 are input from the input unit 40. The blood vessel diameter measuring unit 120 measures changes in the blood vessel diameter of the central artery using ultrasound. Then, the calibrating unit 130 calibrates the parameter related to the blood pressure estimation process for estimating the central blood pressure from the blood vessel diameter of the central artery, using the results of measurement by the blood pressure meter 2 and the blood vessel diameter measuring unit 120 during a given correspondence period, of a one-heartbeat period, in which the relationship between the blood vessel diameter of the central artery and the blood pressure in the peripheral artery corresponds to the relationship between the blood vessel diameter of the central artery and the central blood pressure.

Although it is difficult to noninvasively measure the blood pressure in a central artery, it is easy to noninvasively measure the blood vessel diameter of a central artery. For this reason, in order to estimate the central blood pressure by performing the blood pressure estimation process for estimating the central blood pressure from the blood vessel diameter of the central artery, the parameter related to the blood pressure estimation process is calibrated. Calibration of this parameter usually requires the blood pressure in the central artery, but considering the difficulty of measurement of the blood pressure in the central artery, the parameter is calibrated using the blood pressure in a peripheral artery. Thus, the central blood pressure estimation parameter can be calibrated without the need for measuring the blood pressure in the central artery.

The period setting unit 150 detects a diastolic period after a dicrotic wave peak from among the changes in the blood pressure that have been input from the input unit 40, and sets a part of or the entire period of this diastolic period as a calibration data acquisition period. The diastolic period after the dicrotic wave peak is a period in which the relationship between the carotid artery diameter and the radial artery blood pressure can be regarded as the relationship between the carotid artery diameter and the central blood pressure. Therefore, the central blood pressure estimation parameter can be appropriately calibrated by using the results of measurement of the carotid artery diameter and the radial artery blood pressure measurement during this period.

Moreover, the synchronizing unit 160 performs the synchronization process for synchronizing the changes in the blood pressure that have been input from the input unit 40 with the changes in the blood vessel diameter that have been measured by the blood vessel diameter measuring unit 120. Then, the calibrating unit 130 calibrates the central blood pressure estimation parameter using the results of measurement of the blood pressure and the blood vessel diameter synchronized by the synchronizing unit 160. This makes it possible to accurately perform calibration of the central blood pressure estimation parameter, taking the delay in pulse wave propagation into account.

### 3. Variations

Examples to which the invention can be applied are not limited to the foregoing example, and it goes without saying that appropriate changes can be made without departing from the gist of the invention. The following is a description of variations.

3-1. Blood Vessel Cross Section Index Value

In the above-described embodiment, a case where blood vessel diameter is used as the blood vessel cross section index value has been described. However, blood vessel cross-sectional area may also be used as the blood vessel cross section index value. Correlation characteristics between the blood vessel cross-sectional area and the blood pressure can be defined in the same manner as described above by replacing the blood vessel diameter “D” in Formula 1 with the blood vessel cross-sectional area “S”. The blood vessel cross-sectional area can be, for example, obtained by tracing from a B-mode image or obtained from a color Doppler blood flow display.

Moreover, the method of blood vessel cross section index value measurement is not at all limited to the method in which measurement is performed using ultrasound. As another method, for example, the blood vessel cross section index value may also be measured using a method in which measurement is performed using light. In this case, the blood vessel cross section index value of the target artery can be measured by emitting a light beam of a predetermined wavelength from a light emitting element toward the target artery, receiving reflected light from the target artery, and performing signal processing.

3-2. Correlation Characteristics and Central Blood Pressure Estimation Parameter

In the above-described embodiment, a case where the correlation formula expressed by Formula 1 is applied as the correlation characteristics between blood vessel diameter and blood pressure has been described as an example. However, the correlation formula of Formula 1 is given as an example only, and it goes without saying that any correlation formula other than this may also be applied. With regard to the type of the correlation formula, both linear and nonlinear correlation formulae are applicable.

For example, a correlation formula expressed by Formula 2 below may also be applied as a correlation formula in which the blood vessel diameter and the blood pressure are approximated by a linear relationship:

P=E×D+B (2)

where E=(Ps−Pd)/(Ds−Dd) and B=Pd−E×Dd.

Here, “Ps” represents the systolic blood pressure, and “Pd” represents the diastolic blood pressure. Moreover, “Ds” represents the systolic blood vessel diameter, and “Dd” represents the diastolic blood vessel diameter. Furthermore, “E” represents an elastic modulus that indicates the elasticity of a blood vessel, and “B” represents an intercept of the correlation formula.

In the case where the blood pressure estimation process is performed by applying Formula 2, the value of the elastic modulus “E” in Formula 2 can be used as the central blood pressure estimation parameter, and the value of the elastic modulus “E” can be specified in the same manner as in the above-described embodiment.

3-3. Setting of Calibration Data Acquisition Period

The calibration process in FIG. 6 is a process in which a calibration data acquisition period is set based on (A) the diastolic blood pressure stable change period, which has been mentioned in the description of the principle. However, it goes without saying that the calibration process may also be a process in which the calibration data acquisition period is set based on (B) the blood pressure rising period or (C) the diastolic blood pressure stable change period+the blood pressure rising period.

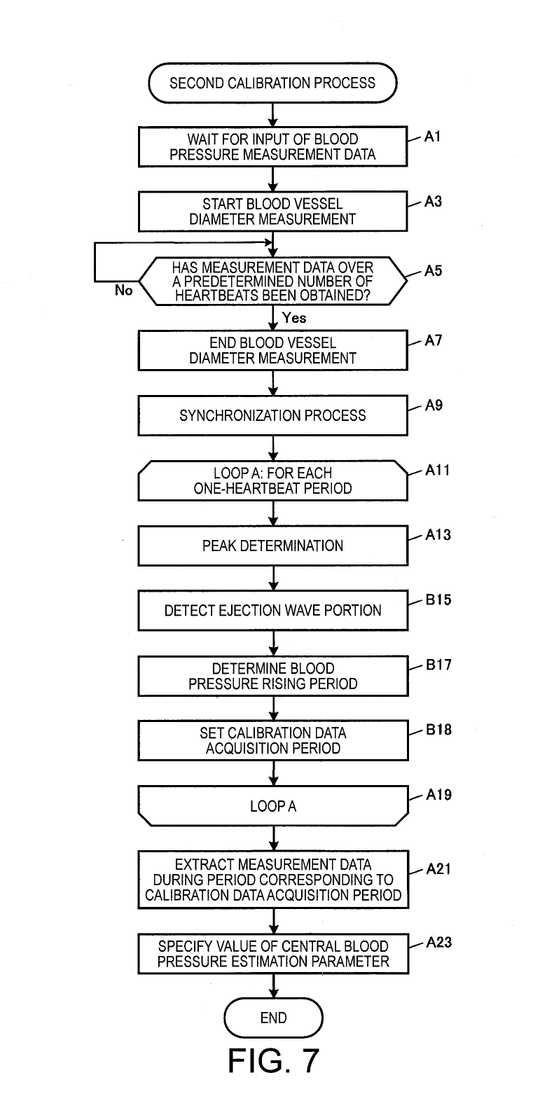

FIG. 7 is a flowchart illustrating the flow of a second calibration process that the processing unit 100 of the foregoing example executes in this case instead of the calibration process in FIG. 6. It should be noted that the same steps as those of the calibration process will be denoted by the same reference numerals, and a repetitive description thereof will be omitted.

In the processing of the loop A, after the peak determination is performed (step A13), the period setting unit 150 detects a portion from the diastolic blood pressure to the ejection wave peak within the one-heartbeat period as an ejection wave portion (step B15).

Subsequently, the period setting unit 150 determines a blood pressure rising period (step B17). Specifically, the elapsed time is calculated from when the diastolic blood pressure is measured until when the ejection wave peak is measured. Then, based on the calculated elapsed time, for example, a period from the start of rising of the ejection wave portion to the time when ⅓ of that ejection wave portion elapses is determined as the blood pressure rising period.

Then, the period setting unit 150 sets a calibration data acquisition period so as to contain at least the blood pressure rising period determined in step B17 (step B18). Then, the processing unit 100 advances the processing to the subsequent one-heartbeat period.

Moreover, in addition to the method that has been given in the above-described embodiment, there are also variations of the method for setting the calibration data acquisition period. For example, rather than setting the entirety of the diastolic blood pressure stable change period as the calibration data acquisition period, it is also possible to set a period corresponding to a latter half portion of the diastolic blood pressure stable change period (for example, a period of the diastolic blood pressure stable change period after the lapse of ½ thereof) as the calibration data acquisition period. Moreover, rather than setting the entirety of the diastolic blood pressure stable change period+the blood pressure rising period as the calibration data acquisition period, it is also possible to set the calibration data acquisition period so as to straddle the border between the diastolic blood pressure stable change period and the blood pressure rising period.

That is to say, it is only required that the calibration data acquisition period is set so as to contain at least a part or the entirety of a diastolic period after the dicrotic wave peak, or that the calibration data acquisition period is set so as to contain at least the blood pressure rising period of the ejection wave portion. The manner in which the calibration data acquisition period is set can be changed as appropriate without departing from the scope of the principle.

Moreover, it is not necessarily required that the blood pressure rising period is set to a period from the start of rising of the ejection wave portion to when ⅓ of that ejection wave portion elapses. Based on the knowledge of the inventor of the invention, it is preferable that a period from the start of rising of the ejection wave portion to when ⅕ to ⅓ of that ejection wave portion elapses is set as the blood pressure rising period. For this reason, for example, a period from the start of rising of the ejection wave portion to when ⅕ of that ejection wave portion elapses may also be set as the blood pressure rising period.

3-4. Central Blood Pressure Measuring Apparatus

In the foregoing example, the blood pressure measuring apparatus that measures central blood pressure has been described as a type of ultrasonic blood pressure meter that the subject wears around the neck during usage. However, this configuration is merely an example. In addition to this, the body apparatus may be configured so as to be wrapped around a brachial portion of the subject during usage, or the body apparatus may be configured so as to be worn on the wrist of the subject during usage. Moreover, it is not necessarily required that the ultrasound probe and the body apparatus are separate from each other, and a blood pressure measuring apparatus may also be configured in which the ultrasound probe and the body apparatus are provided in the same housing.

Furthermore, in the above-described embodiment, an embodiment of the blood pressure measuring apparatus that is designed to enable an ambulant subject to measure central blood pressure by himself/herself has been described. However, the blood pressure measuring apparatus to which the invention is applicable is not limited to this. For example, the invention is also applicable to a medical blood pressure measuring apparatus that enables a technician to perform ultrasound diagnosis of a subject in a lying position using an ultrasound probe.

3-5. Peripheral Artery Blood Pressure Measuring Apparatus

The foregoing example has been described using a tonometer as an example of the blood pressure measuring apparatus for calibration. However, this also is merely an example. For example, a cuff-type sphygmomanometer may also be used instead of the tonometer. Generally, cuff-type sphygmomanometers are not capable of obtaining a continuous blood pressure waveform. However, in the oscillometric method, the volume of a blood vessel changes as the cuff pressure is reduced, and blood pressure is determined from minute pressure fluctuations that occur in the cuff in accordance with the changes in the volume of the blood vessel. Thus, changes in blood pressure in a peripheral artery can be measured by configuring the apparatus so as to acquire a volume fluctuation waveform from the sphygmomanometer and converting the acquired volume fluctuation waveform into a blood pressure waveform. In this case, the peripheral artery used for blood pressure measurement is not necessarily required to be the radial artery, and may also be the brachial artery.

3-6. Communication System

In the above-described embodiment, the ultrasonic blood pressure meter 1 and the blood pressure meter 2 are connected to each other in a wired manner. However, a configuration is also possible in which the ultrasonic blood pressure meter 1 and the blood pressure meter 2 are equipped with respective wireless communication units, and the measured values of blood pressure are acquired from the blood pressure meter 2 by means of wireless communication.

The entire disclosure of Japanese Patent Application No. 2012-157225, filed Jul. 13, 2012 is expressly incorporated by reference herein.

What is claimed is:

1. A blood pressure measuring apparatus comprising:
an input unit from which changes in blood pressure in a peripheral artery that are measured by a blood pressure measuring apparatus are input;
a blood vessel cross section index value measuring unit that measures changes in a blood vessel cross section index value that is a blood vessel diameter or a blood vessel cross-sectional area of a central artery; and
a calibrating unit that calibrates a parameter related to a blood pressure estimation process for estimating central blood pressure from the blood vessel cross section index value, using results of measurement by the blood vessel cross section index value measuring unit.

2. The blood pressure measuring apparatus according to claim 1, further comprising:
a first period setting unit that detects a diastolic period after a dicrotic wave peak from among the changes in the blood pressure that are input from the input unit, and sets the first period so as to contain at least a part or the entirety of the diastolic period.

3. The blood pressure measuring apparatus according claim 1, further comprising:
a second period setting unit that detects an ejection wave portion from among the changes in the blood pressure that are input from the input unit, and sets the second period so as to contain at least a given rising period of the ejection wave portion.

4. The blood pressure measuring apparatus according to claim 3,
wherein the second period setting unit performs setting such that the rising period contains at least a period from a start of rising of the ejection wave portion to when ⅕ of the ejection wave portion elapses.

5. The blood pressure measuring apparatus according to claim 1, further comprising:
a synchronizing unit that synchronizes the changes in the blood pressure that are input from the input unit with the changes in the blood vessel cross section index value that are measured by the blood vessel cross section index value measuring unit,
the calibrating unit calibrating the parameter using measurement results of the blood pressure and the blood vessel cross section index value that are synchronized by the synchronizing unit.

6. A method for calibrating a central blood pressure estimation parameter, comprising:
measuring changes in blood pressure in a peripheral artery;
measuring changes in a blood vessel cross section index value of a central artery; and
calibrating a parameter related to a blood pressure estimation process for estimating central blood pressure from the blood vessel cross section index value, using the blood vessel cross section index value.
